(19) United States
(12) Patent Application Publication
(10) Pub. No.: US 2022/0143119 A1
(43) Pub. Date: May 12, 2022

(54) CROTON LECHLERI COMPOSITIONS FOR USE IN THE TREATMENT OF SKIN CANCER

(71) Applicant: ALPHYN BIOLOGICS, LLC, Cincinnati, OH (US)

(72) Inventors: Gary Michael PEKOE, Cincinnati, OH (US); Jazmyne Kristyne MINK, Cincinnati, OH (US); Steven Aaron PENTELNIK, Cincinnati, OH (US); Neal G. KOLLER, Annapolis, MD (US)

(21) Appl. No.: 17/599,495

(22) PCT Filed: Mar. 20, 2020
(86) PCT No.: PCT/US2020/024066

§ 371 Date: Sep. 28, 2021

Related U.S. Application Data
(60) Provisional application No. 62/821,240, filed on Mar. 20, 2019.

(51) Int. Cl. A61K 36/47; A61K 9/00; A61K 31/353; A61P 35/00

(52) U.S. Cl. CPC A61K 36/47; A61P 35/00; A61K 31/353; A61K 9/0014

(57) ABSTRACT

The present disclosure provides for the treatment of precancerous or cancerous skin lesions via the topical administration of a pharmaceutical composition comprising a therapeutically effective amount of an extract of the Croton lechleri tree. Also provided are details of studies on the effectiveness of an extract of the Croton lechleri tree on precancerous or cancerous skin lesions.

### CROSS-REFERENCE TO RELATED APPLICATIONS

This application claims the benefit of U.S. Provisional Application No. 62/821,240 filed Mar. 20, 2019. The disclosure of the application is incorporated herein by reference.

### SUMMARY

The present invention is generally related to the treatment of precancerous or cancerous skin lesions via the topical administration of a pharmaceutical compositions comprising a therapeutically effective amount of latex of Croton lechleri, preferably filtered latex of Croton lechleri, preferably filtered latex of Croton lechleri Müll.Arg.

### BRIEF DESCRIPTION OF THE DRAWINGS

FIG. 1 depicts a representative Total Ion Chromatogram as well as additional Multiple Reaction Monitoring spectra that identify the marker compounds in an AB-101 composition.

FIG. 2A depicts the NMR spectra of 3 lots of AB-101 in D₂O—the top spectra is for Lot 00, the middle spectra is for Lot 01, and the bottom spectra is for Lot 02.

FIG. 2B depicts the overlay of the NMR spectra of Lots 00, 01, and 02 of AB-101 in D₂O.

FIG. 3A depicts the Nuclear Magnetic Resonance (NMR) spectra of 3 lots of AB-101 in d₄-Methanol—the top spectra is for Lot 00, the middle spectra is for Lot 01, and the bottom spectra is for Lot 02.

FIG. 3B depicts the overlay of the NMR spectra of Lots 00, 01, and 02 of AB-101 in d₄-Methanol.

FIG. 4A depicts the NMR spectra of 4 lots of AB-101 in d₄-Methanol—the top spectra is for Lot 00, the upper middle spectra is for Lot 01, the lower middle is for Lot 02, and the bottom spectra is for Lot X.

FIG. 4B depicts the overlay of the NMR spectra of Lots 00, 01, 02, and X of AB-101 in d₄-Methanol.

FIG. 5 depicts bar graphs comparing the AB-101 lot analysis results for A) gallocatechin B) epigallocatechin C) catechin D) epicatechin and E) taspine.

### DEFINITIONS

Before the present compositions and methods are described, it is to be understood that this invention is not limited to the particular processes, compositions, or methodologies described, as these may vary. It is also to be understood that the terminology used in the description is for the purpose of describing the particular versions or embodiments only and is not intended to limit the scope of embodiments herein which will be limited only by the appended claims. Unless specifically defined herein, all technical and scientific terms used herein have the same meanings as commonly understood by one of ordinary skill in the art. Although any methods and materials similar or equivalent to those described herein can be used in the practice or testing of embodiments of embodiments herein, the preferred methods, devices, and materials are now described. All publications mentioned herein are incorporated by reference in their entirety. Nothing herein is to be construed as an admission that embodiments herein are not entitled to antedate such disclosure by virtue of prior invention.

It must also be noted that as used herein and in the appended claims, the singular forms “a,” “an,” and “the” include plural reference unless the context clearly dictates otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean plus or minus 10% of the numerical value of the number with which it is being used. Therefore, about 50% means in the range of 45%-55%.

As used herein, the term “AB-101” maybe used interchangeably with latex of Croton lechleri, preferably filtered latex of Croton lechleri, preferably filtered latex of Croton lechleri Müll.Arg. The latex is excreted material from the wounded trunk of Croton lechleri, preferably of Croton lechleri Müll.Arg.

“Administering” when used in conjunction with a therapeutic, such as AB-101, means to administer a therapeutic directly into or onto a target tissue or to administer a therapeutic to a patient whereby the therapeutic positively impacts the tissue to which it is targeted. Thus, as used herein, the term “administering”, when used in conjunction with a composition of embodiments herein, can include, but is not limited to, providing the composition into or onto the target tissue; providing the composition to a patient by, e.g., topical application whereby the therapeutic reaches the target tissue. “Administering” a composition may be accomplished topically or in combination with other known techniques.

The transitional term “comprising,” which is synonymous with “including,” “containing,” or “characterized by,” is inclusive or open-ended and does not exclude additional, unrecited elements or method steps.

In embodiments or claims where the term “comprising” is used as the transition phrase, such embodiments can also be envisioned with replacement of the term “comprising” with the terms “consisting of” or “consisting essentially of.”

As used herein, the term “consists of” or “consisting of” means that the pharmaceutical composition, composition or the method includes only the elements, steps, or ingredients specifically recited in the particular claimed embodiment or claim.

As used herein, the term “consisting essentially of” or “consists essentially of” means that the pharmaceutical composition, or the method includes only the elements, steps or ingredients specifically recited in the particular claimed embodiment or claim and may optionally include additional elements, steps or ingredients that do not materially affect the basic and novel characteristics of the particular embodiment or claim. For example, the only active ingredient(s) in the composition or method that treats the specified condition (e.g., nutrient depletion) is the specifically recited therapeutic(s) in the particular embodiment or claim.

The term “combination therapy” means the administration of two or more therapeutic agents to treat a condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “excipient” and “pharmaceutically acceptable excipient” as used herein are intended to be generally synonymous, and is used interchangeably with, the terms “carrier,” “pharmaceutically acceptable carrier,” “diluent,” “pharmaceutically acceptable diluent.”

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

As used herein, the term “pharmaceutically acceptable salt” refers to a salt prepared from a base or acid which is acceptable for administration to a patient, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

As used herein, the term “therapeutic” or “therapeutic agent” or “pharmaceutically active agent” means an agent utilized to treat, combat, ameliorate, prevent or improve an unwanted condition or disease of a patient. In part, embodiments of the present invention are directed to the treatment of precancerous or cancerous skin lesions, including, but not limited to, Non-hypertrophic Actinic Keratosis (AK), Superficial Basal Cell Carcinoma, Nodular Basal Cell Carcinoma, Superficial Squamous Cell Carcinoma, Squamous Cell Carcinoma, Squamous Cell Carcinoma in situ, and Mycosis Fungoides.

The term “therapeutically acceptable” refers to those compositions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compositions disclosed herein which are water or oil-soluble or dispersible and therapeutically acceptable as defined herein. The salts can be prepared during the final isolation and purification of the compositions or separately by reacting the appropriate composition in the form of the free base with a suitable acid.

The phrase “therapeutically effective” is intended to qualify the amount of active ingredients used in the treatment of a disease or disorder or on the effecting of a clinical endpoint.

A “therapeutically effective amount” or “effective amount” of a composition is a predetermined amount calculated to achieve the desired effect, i.e., but not limited to, to inhibit, block, or reverse the activation, migration, or proliferation of cells. The activity contemplated by the present methods includes both medical therapeutic and/or prophylactic treatment, as appropriate. The specific dose of a composition administered according to this invention to obtain therapeutic and/or prophylactic effects will, of course, be determined by the particular circumstances surrounding the case, including, for example, the composition administered, the route of administration, and the condition being treated. The compositions are effective over a wide dosage range and, for example, dosages per application will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of composition to be administered, and the chosen route of administration, and therefore the above dosage ranges are not intended to limit the scope of the invention in any way. A therapeutically effective amount of the composition of this invention is typically an amount such that when it is administered in a physiologically tolerable excipient composition, it is sufficient to achieve an effective systemic concentration or local concentration in the tissue.

The terms “treat,” “treated,” “treating”, or “treatment” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “topical” includes administering to any skin or mucosal surface or being suitable for such administration. In some embodiments, “topical” may be the skin surface. Skin surface includes any part of the body, including but not limited to face, hands, legs, neck, abdominal area, eyes, nose, and chest. Mucosal surface includes, without limitation, mucosa of the mouth or oral mucosa, lips, tongue, nasal, buccal mucosa, palate, gingiva, nasopharynx, respiratory epithelium, conjunctiva, vagina, cervix, and urethral mucosa.

Also provided are embodiments wherein any embodiment herein may be combined with any one or more of the other embodiments, unless otherwise stated and provided the combination is not mutually exclusive.

Croton lechleri (a member of the family Euphorbiaceae, commonly called the spurge family) has approximately 1,300 species of plants that are either herbaceous (plants that have no persistent woody stem above ground), shrub (a woody plant which is smaller than a tree and has several main stems arising at or near the ground), tree (a perennial plant with an elongated stem, or trunk, supporting branches and leaves in most species), or liana (any of various long-stemmed, woody vines that are rooted in the soil at ground level and use trees, as well as other means of vertical support, to climb up to the canopy to get access to well-lit areas of the forest) forms. The Croton genus is a diverse and complex group of flowering plants ranging from herbs and shrubs to trees, The Croton genus is widely distributed in tropical and subtropical regions around the world.

Dragon's blood refers to a bright red resin that is obtained from different species of a number of distinct plant genus: Croton, Dracaena, Daemonorops, Calamus rotang and Pterocarpus. The red resin has been in continuous use since ancient times as varnish, medicine, incense, and dye. The name dragon's blood is used to refer to all of the above plant genus, often without any distinction as to the genus or species it is coming from. Those with the same genus will be similar in any therapeutic or nutritional value, with factors such as local soil, local rainfall, local humidity, local sunlight, local fauna and the like imparting variability and inconsistency. However, the difference between the red resin coming from Croton versus Daemonorops (a genus of rattan palms in the family Arecaceae found primarily in the tropics and subtropics of southeastern Asia with a few species extending into southern China and the Himalayas) will be significant. The Croton and Daemonorops genus originate from opposite sides of the world so their components are different and therefore specificity of source plant is important to deliver the desired medicinal benefits or avoid undesirable toxic results. For example milky white latex that is often toxic or at least irritating to the skin is common to the members of the spurge or Euphorbiaceae family Therefore selecting the specific genus, species, and local geographical area of the spurge or Euphorbiaceae family is essential to having the possibility for latex to have specific and repetitive medicinal properties.

A handful of Croton species found in the South America rainforest (in countries of Bolivia, Brazil, Colombia, Ecuador and Peru) Central America and Mexico produce the red latex, commonly known as dragon's blood, that has medicinal properties. The dragon's blood trees grown in these areas include Croton lechleri, Croton draco, Croton palanostigma, Croton sordidus, Croton urucurana, and Croton xalapenesis.

While the desired medicinal properties could be found by extracting the compositions from either the leaves or bark, in preferred embodiments, it is the deep red latex of Croton lechleri, preferably filtered latex of Croton lechleri, preferably filtered latex of Croton lechleri Müll.Arg, that is also referred to as latex, that is utilized. According to Langenheim (2003) resin “is a lipid-soluble mixture of volatile and non-volatile terpenoid and/or phenolic secondary compounds that are usually secreted in specialized structures located either internally or on the surface of the plant and are of potential significance in ecological interactions”. By contrast, latex, is a mixture of terpenoids, phenolic compounds, acids, carbohydrates, etc. having a protective role (Lewisohn 1991) and produced in special cells called laticifers (Fahn 1979). Chemical characterization of dragon's blood is species specific and has been undertaken by many authors. For example, it is possible to distinguish between dragon's blood from some individual species used in works of art, since it has been sold as a colorant for many centuries (Baumer and Dietemann 2010). Dragon's blood of Croton spp. is usually referred to as latex due to the fact that it is secreted and stored by laticifers, and its major constituents are polymeric anthocyanidins, which co-occur with many minor constituents, including diterpenes and simple phenols (Salatino et al. 2007). Dragon's blood secreted by stems of Pterocarpus officinalis is also called latex (Weaver 1997; Guerrero and Guzman 2004); however, information about the chemical composition of the exudate and its ecological function is poorly known. Dragon's blood derived from species of Dracaena and Daemonorops is a phenolic resin (Langenheim 2003), with well-recognized chemical content (e.g. Gonzalez et al. 2000; Shen et al. 2007; Sousa et al. 2008). Sometimes, dragon's blood is referred to as latex (e.g. Philipson 2001). However, this could prove to be a source of confusion, since plants produce other exudates referred to by that name, such as xylem latex and phloem latex, which are entirely different in terms of their location, chemical composition and function. The resin is obtained through tapping the tree or other common draining methods. Draining the tree latex has the additional benefit of not having to use complex and costly extraction technology to obtain the desired composition from either the leaves or bark. The latex of Croton lechleri Müll.Arg. of the present application is then filtered in a 30 micron filter to remove plant debris and thick, resinous material. Chemical characterization of dragon's blood is local geography specific and has not been undertaken by prior authors.

Medicinal and toxic properties of various species of the Croton genus have been ascribed to a wide variety of chemical compounds, such as terpenoids and steroids, alkaloids, and phenolic compounds, the latter including predominantly flavonoids, lignans, and proanthocyanidins. Some embodiments of the present application utilize the whole latex, thereby leveraging the “organic” synergy of all the latex components as intended by nature. The molecular classes found in latex of Croton lechleri Müll.Arg. of the present application which provide the desired medicinal benefits of Croton lechleri Müll.Arg. are: Alkaloids, Diterpenes, Lignans, Phenols, Phytosterols, Proanthocyanidins, Sterols and Tannins.

In certain embodiments, the specific dragon's blood tree of the present application is Croton lechleri Müll.Arg. of the Family: Euphorbiaceae. Dragon's blood is also referred to as Sangre de drago (Peru), Sangre de grado (Ecuador). Embodiments of the present invention are directed to pharmaceutical compositions of latex of Croton lechleri, preferably filtered latex of Croton lechleri, preferably filtered latex of Croton lechleri Müll.Arg, wherein the pharmaceutical composition does not contain a pharmaceutically acceptable excipient. Embodiments of the present invention are directed to pharmaceutical compositions of latex of Croton lechleri, preferably filtered latex of Croton lechleri, preferably filtered latex of Croton lechleri Müll.Arg and a pharmaceutically acceptable excipient. Such pharmaceutical compositions have been found to be useful in the successful treatment of precancerous or cancerous skin lesions and methods of using the same. In some embodiments the pharmaceutical compositions are administered topically. Embodiments are directed to pharmaceutical compositions comprising latex of Croton lechleri, preferably filtered latex of Croton lechleri, preferably filtered latex of Croton lechleri disclosed herein together with a pharmaceutically acceptable carrier, as well as methods of making and using the compositions. Certain embodiments are directed to methods for inhibiting precancerous or cancerous skin lesions. Other embodiments are directed to methods for treating precancerous or cancerous skin lesions in a patient in need of such treatment, comprising administering to said patient a therapeutically effective amount of a composition according to the present invention. Also provided is the use of certain extracts of Croton lechleri disclosed herein in the manufacture of a medicament for the treatment of precancerous or cancerous skin lesions.

### Pharmaceutical Compositions

Embodiments herein are directed to pharmaceutical compositions comprising latex of Croton lechleri, preferably filtered latex of Croton lechleri, preferably filtered latex of Croton lechleri Müll.Arg, wherein the pharmaceutical composition does not contain a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, and taspine, and combinations thereof. Each of gallocatechin, epigallocatechin, catechin, epicatechin, and taspine, may be present in the latex of Croton lechleri, preferably filtered latex of Croton lechleri, preferably filtered latex of Croton lechleri Müll.Arg. in the amounts found in Table 1 or paragraphs [0040]-[0045].

Embodiments herein are directed to pharmaceutical compositions comprising latex of Croton lechleri, preferably filtered latex of Croton lechleri, preferably filtered latex of Croton lechleri Müll.Arg and a pharmaceutically acceptable excipient. In certain embodiments, latex of Croton lechleri, preferably filtered latex of Croton lechleri, preferably filtered latex of Croton lechleri Müll.Arg. comprises one or more compounds selected from: gallocatechin, epigallocatechin, catechin, epicatechin, and taspine, and combinations thereof. Each of gallocatechin, epigallocatechin, catechin, epicatechin, and taspine, may be present in the latex of Croton lechleri, preferably filtered latex of Croton lechleri, preferably filtered latex of Croton lechleri Müll.Arg. in the amounts found in Table 1 or paragraphs [0040]-[0045].

TABLE 1

| | Exemplary | Other | |
|---|---|---|---|
| | Amount | Embodiments | Exemplary |
| | present in | Amount | Amount |
| | the latex | Present in the | present |
| | (PPM | latex (PPM | in the latex |
| Compound | is in μg/g) | is in μg/g) | (PPM is in μg/g) |
| Gallocatechin | at least about | at least about | At least about 85 PPM, |
| | 85 PPM | 110 PPM | but less than about 145 |
| Epigallocatechin | at least about | at least about | At least about 300, but |
| | 300 PPM | 780 PPM | less than about 1230 |
| Catechin | at least about | at least about | |
| | 2.4 PPM | 1.6 PPM | |
| Epicatechin | at least about | at least about | |
| | 3.1 PPM | 2 PPM | |
| Taspine | at least about | at least about | |
| | 35 PPM | 45 PPM | |

If the latex of Croton lechleri, preferably filtered latex of Croton lechleri, preferably filtered latex of Croton lechleri Müll.Arg. fails to contain the amounts of gallocatechin, epigallocatechin, catechin, epicatechin, and taspine at least the amounts set forth in Table 1, it is not suitable for use in the pharmaceutical compositions and methods described herein.

In some embodiments, the gallocatechin is in an amount of at least about at least about 85 mg, least about 90 mg, 100 mg, at least about 110 mg, at least about 115 mg, at least about 120 mg, at least about 125 mg, at least about 130 mg, at least about 135 mg, at least about 140 mg, at least about 145 mg, at least about 150 mg, at least about 155 mg, at least about 160 mg, at least about 165 mg, at least about 170 mg, at least about 175 mg, at least about 180 mg, at least about 185 mg, at least about 190 mg, at least about 195 mg, at least about 200 mg, or a range between any two of these values.

In some embodiments, the epigallocatechin is in an amount of at least about 300 mg, at least about 310 mg, at least about 320 mg, at least about 330 mg, at least about 340 mg, at least about 350 mg, at least about 360 mg, at least about 370 mg, at least about 380 mg, at least about 390 mg, at least about 400 mg, at least about 410 mg, at least about 420 mg, at least about 430 mg, at least about 440 mg, at least about 450 mg, at least about 460 mg, at least about 470 mg, at least about 480 mg, at least about 490 mg, at least about 500 mg, at least about 510 mg, at least about 520 mg, at least about 530 mg, at least about 540 mg, at least about 550 mg, at least about 560 mg, at least about 570 mg, at least about 580 mg, at least about 590 mg, at least about 600 mg, at least about 610 mg, at least about 620 mg, at least about 630 mg, at least about 640 mg, at least about 650 mg, at least about 660 mg, at least about 670 mg, at least about 680 mg, at least about 690 mg, at least about 700 mg, at least about 710 mg, at least about 720 mg, at least about 730 mg, at least about 740 mg, at least about 750 mg, at least about 760 mg, at least about 770 mg, at least about 780 mg, at least about 790 mg, at least about 800 mg, at least about 810 mg, at least about 820 mg, at least about 830 mg, at least about 840 mg, at least about 850 mg, at least about 860 mg, at least about 870 mg, at least about 880 mg, at least about 890 mg, at least about 900 mg, at least about 910 mg, at least about 920 mg, at least about 930 mg, at least about 940 mg, at least about 950 mg, at least about 960 mg, at least about 970 mg, at least about 980 mg, at least about 990 mg, at least about 1000 mg, at least about 1010 mg, at least about 1020 mg, at least about 1030 mg, at least about 1040 mg, at least about 1050 mg, at least about 1060 mg, at least about 1070 mg, at least about 1080 mg, at least about 1090 mg, at least about 1100 mg, at least about 1110 mg, at least about 1120 mg, at least about 1130 mg, at least about 1140 mg, at least about 1150 mg, at least about 1160 mg, at least about 1170 mg, at least about 1180 mg, at least about 1190 mg, at least about 1200 mg, at least about 1210 mg, at least about 1220 mg, at least about 1230 mg, at least about 1240 mg, at least about 1250 mg, at least about 1260 mg, at least about 1270 mg, at least about 1280 mg, at least about 1290 mg, at least about 1300 mg, at least about 1310 mg, at least about 1320 mg, at least about 1330 mg, at least about 1340 mg, at least about 1350 mg, at least about 1360 mg, at least about 1370 mg, at least about 1380 mg, at least about 1390 mg, at least about 1400 mg, at least about 1410 mg, at least about 1420 mg, at least about 1430 mg, at least about 1440 mg, at least about 1450 mg, at least about 1460 mg, at least about 1470 mg, at least about 1480 mg, at least about 1490 mg, at least about 1500 mg, at least about 1510 mg, at least about 1520 mg, at least about 1530 mg, at least about 1540 mg, at least about 1550 mg, at least about 1560 mg, at least about 1570 mg, at least about 1580 mg, at least about 1590 mg, at least about 1600 mg, at least about 1610 mg, at least about 1620 mg, at least about 1630 mg, at least about 1640 mg, at least about 1650 mg, at least about 1660 mg, at least about 1670 mg, at least about 1680 mg, at least about 1690 mg, at least about 1700 mg, or a range between any two of these values.

In some embodiments, the catechin is in an amount of at least about 1.6 mg, at least about 1.7 mg, at least about 1.8 mg, at least about 1.9 mg, at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, at least about 10.1 mg, at least about 10.2 mg, at least about 10.3 mg, at least about 10.4 mg, at least about 10.5 mg, at least about 10.6 mg, at least about 10.7 mg, at least about 10.8 mg, at least about 10.9 mg, at least about 11.0 mg, or a range between any two of these values.

In some embodiments, the epicatechin is in an amount of at least about 2.0 mg, at least about 2.1 mg, at least about 2.2 mg, at least about 2.3 mg, at least about 2.4 mg, at least about 2.5 mg, at least about 2.6 mg, at least about 2.7 mg, at least about 2.8 mg, at least about 2.9 mg, at least about 3.0 mg, at least about 3.1 mg, at least about 3.2 mg, at least about 3.3 mg, at least about 3.4 mg, at least about 3.5 mg, at least about 3.6 mg, at least about 3.7 mg, at least about 3.8 mg, at least about 3.9 mg, at least about 4.0 mg, at least about 4.1 mg, at least about 4.2 mg, at least about 4.3 mg, at least about 4.4 mg, at least about 4.5 mg, at least about 4.6 mg, at least about 4.7 mg, at least about 4.8 mg, at least about 4.9 mg, at least about 5.0 mg, at least about 5.1 mg, at least about 5.2 mg, at least about 5.3 mg, at least about 5.4 mg, at least about 5.5 mg, at least about 5.6 mg, at least about 5.7 mg, at least about 5.8 mg, at least about 5.9 mg, at least about 6.0 mg, at least about 6.1 mg, at least about 6.2 mg, at least about 6.3 mg, at least about 6.4 mg, at least about 6.5 mg, at least about 6.6 mg, at least about 6.7 mg, at least about 6.8 mg, at least about 6.9 mg, at least about 7.0 mg, at least about 7.1 mg, at least about 7.2 mg, at least about 7.3 mg, at least about 7.4 mg, at least about 7.5 mg, at least about 7.6 mg, at least about 7.7 mg, at least about 7.8 mg, at least about 7.9 mg, at least about 8.0 mg, at least about 8.1 mg, at least about 8.2 mg, at least about 8.3 mg, at least about 8.4 mg, at least about 8.5 mg, at least about 8.6 mg, at least about 8.7 mg, at least about 8.8 mg, at least about 8.9 mg, at least about 9.0 mg, at least about 9.1 mg, at least about 9.2 mg, at least about 9.3 mg, at least about 9.4 mg, at least about 9.5 mg, at least about 9.6 mg, at least about 9.7 mg, at least about 9.8 mg, at least about 9.9 mg, at least about 10.0 mg, or a range between any two of these values.

In some embodiments, the taspine is in an amount of 35 mg, an amount of 40 mg, an amount of 45 mg, at least about 46 mg, at least about 47 mg, at least about 48 mg, at least about 49 mg, 50 mg, at least about 51 mg, at least about 52 mg, at least about 53 mg, at least about 54 mg, at least about 55 mg, at least about 56 mg, at least about 57 mg, at least about 58 mg, at least about 59 mg, at least about 60 mg, at least about 61 mg, at least about 62 mg, at least about 63 mg, at least about 64 mg, at least about 65 mg, or a range between any two of these values.

The pharmaceutical composition of AB-101 as described and claimed herein is a plant sourced material that meets the criteria of being consistently reproducible between batch to batch and reliably delivers the desired health benefits via topical application that may be used in a pharmaceutical composition. It can be used to treat skin conditions associated with Non-hypertrophic Actinic Keratosis (AK), referred to as pre-cancer, Basal Cell Carcinoma (BCC) inclusive of superficial BCC and nodular BCC and Squamous Cell Carcinoma (SCC) inclusive of superficial SCC and SCC in situ. Plant sourced materials face the challenge that changes in environmental weather, climate, rainfall, time of harvest (via season, time of day or month), changes in geography, longitude location, latitude location, altitude, changes in soil condition, harvesting protocols and many additional conditions can alter the characteristics of the plant that could impact quality. This can impact the plant's bioactivity resulting in inconsistency in achieving desired performance outcome. This creates a challenge in defining a pharmaceutical grade of dragon's blood to deliver consistent and reproducible therapeutic benefits. This is further compounded by the wide variety of the different species called dragon's blood. For example, Croton lechleri latex and isolated alkaloid taspine exhibits inhibition against human melanoma SK23. In contrast, AB-101 did not show effectiveness in topical melanoma. The generic name of the Croton lechleri resin, ie, dragon's blood, or Sangre de grado, creates confusion in defining a plant-derived pharmaceutical and demonstrates that not all Croton lechleri plants are the same, nor do they provide similar benefits. This unexpected result also shows that not all cancer cell activity translates to in use benefits. This is the reason that AB-101 101 (latex of Croton lechleri, preferably filtered latex of Croton lechleri, preferably filtered latex of Croton lechleri Müll.Arg. with the appropriate levels of gallocatechin, epigallocatechin, catechin, epicatechin, and taspine) skin cancer benefits focused on human use applications.

The benefits of AB-101, filtered or unfiltered latex of Croton lechleri, preferably filtered latex of Croton lechleri, preferably filtered latex of Croton lechleri Müll.Arg. is it's ability to deliver consistent results for treating specific types of pre-cancerous skin conditions and skin cancers between batch to batch in spite of all the confounding conditions. The challenge in using the whole latex is to identify compounds that delivers performance based on the many bio-active compounds comprising the latex. Even within the same species, grown in a similar location, there are variations in chemical content and bioactivity of the whole latex that unexpectedly varies in its ability to fight and kill cancer.

Methodology that can identify the whole latex is effective by having an assay that determines when a batch meets the predetermined performance criteria. Having a unique analytical and microbiological assay enables the ability to identify which batch of filtered or unfiltered latex of Croton lechleri, preferably filtered latex of Croton lechleri, preferably filtered latex of Croton lechleri Müll.Arg, has the combination of components that will consistently deliver the desired outcome.

AB-101 botanical raw material (BRM) is a complex botanical product that is a latex of Croton lechleri, preferably filtered latex of Croton lechleri, preferably filtered latex of Croton lechleri Müll.Arg. that contains certain marker compounds (catechin, gallocatechin, epicatechin, epigallocatechin, and taspine) in specified amounts (see Table 1). Utilization of latex of Croton lechleri, preferably filtered latex of Croton lechleri, preferably filtered latex of Croton lechleri Müll.Arg. can be used to characterize the existence and levels of such marker compounds for batch to batch consistency and repeatable performance of AB-101. Marker compounds in AB-101 BRM include the proanthocyanidins: catechin, gallocatechin, epicatechin, and epigallocatechin, as well as the alkaloid taspine.

The published and accepted taxonomic classification of Croton lechleri is the following (van Ee & Berry, 2011, Riina et al, 2009, The Plant List, 2012, The Angiosperm Phylogeny Group, 2009):

```
Division: Streptophyta
  Class: Equisetopsida
    Subclass: Magnoliidae
      Order: Malpighiales
        Family: Euphorbiaceae
          Genus: Croton
            Subgenus Adenophylli
              Section: Cyclostigma
                Subsection: Cyclostigma
                  Species: Crown lechleri Müll.Arg.
```

Biodiversity of botanicals plays a major role in constituent chemical compound characterization. Chemical compounds utilized for as important batch to batch consistency of AB-101 need to 1) demonstrate antimicrobial or cicatrizant properties, 2) be present in AB-101, and 3) be detectable using analytical techniques. Using these criteria, the analytical efforts focused on 2 classes of compounds: polyphenols (proanthocyanidins) and alkaloids (taspine). Within the proanthocyanidin class, 4 specific compounds were focused on: catechin, epicatechin, gallocatechin, and epigallocatechin. The compound of importance within the alkaloid class is taspine. Each of these compounds fulfills the three required elements detailed above. The following are the chemical structures of the 5 compounds utilized as important markers for batch to batch consistency of AB-101.

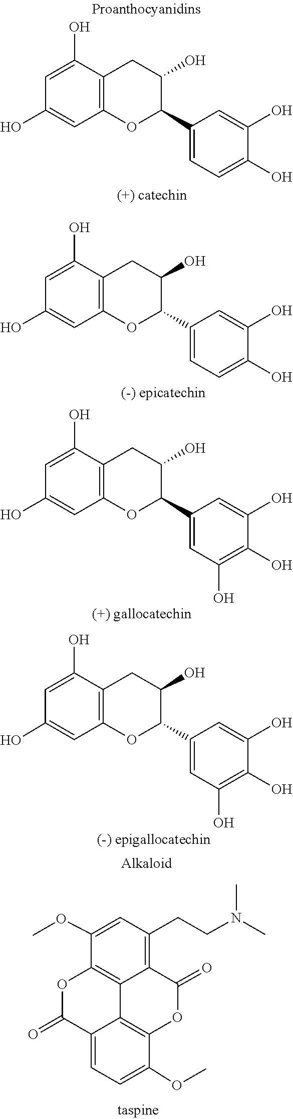

For characterization studies, AB-101 extract was lyophilized and the lyophilized powder was subjected to three different extraction methods.

Method 1—Ultrasonic polyphenol extraction. The lyophilized AB-101 extract was dissolved into methanol. The resultant emulsion was then subjected to sonication for 10 minutes followed by centrifugation to remove particulates for 5 minutes. The supernatant was then subjected to LC-MS/MS analysis.

Method 2—Soxhlet extraction. The lyophilized AB-101 extract was subjected to a Soxhlet extraction with 80% ethanol. The ethanol was removed via a rotary evaporator. The resultant material was then subjected re-suspended in ethanol then subjected to LC-MS/MS analysis.

Method 3—Polyphenol extraction. The lyophilized AB-101 extract was incubated with methanol overnight at room temperature and in the dark. The supernatant was then filtered using Whatman filters, dried, and then re-suspended in methanol. The resultant material was then subjected to LC-MS/MS analysis.

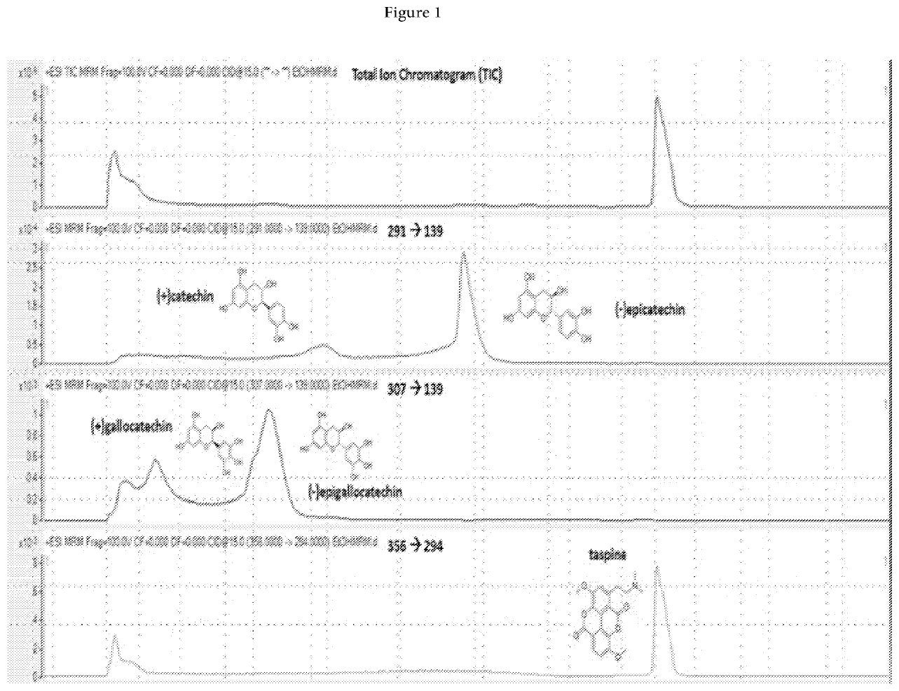

FIG. 1 depicts a representative Total Ion Chromatogram as well as additional Multiple Reaction Monitoring spectra that identify the important marker compounds in an AB-101 extract. The compounds are detectable using any of the three extraction methods.

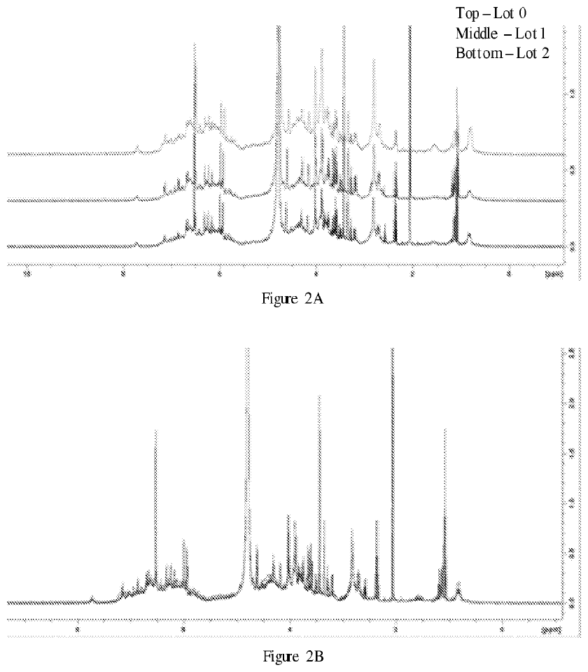

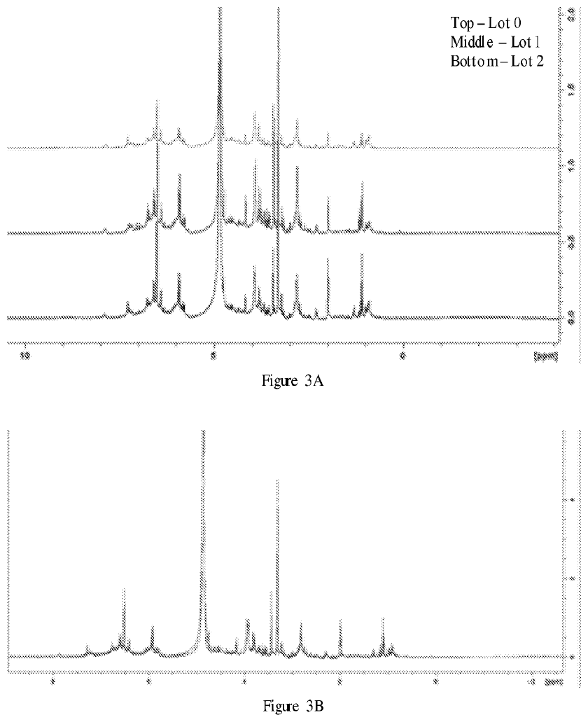

Biodiversity contributes to vast amounts of variability. In order to capture this variability, an NMR method utilizing a “spectral fingerprint” was used with an overlapping a reference standard. These fingerprinting captures most components within AB-101 and would be quantifiable using Nuclear Magnetic Resonance (NMR). Examples of NMR spectra using three different AB-101 lots (Lots 00, 01, and 02 respectively) and two different deuterated solvents (D₂O and d₄-Methanol respectively) are shown in FIGS. 2A and 3A with overlays of each solvents spectra being shown in FIGS. 2B and 3B and demonstrated no significant variability.

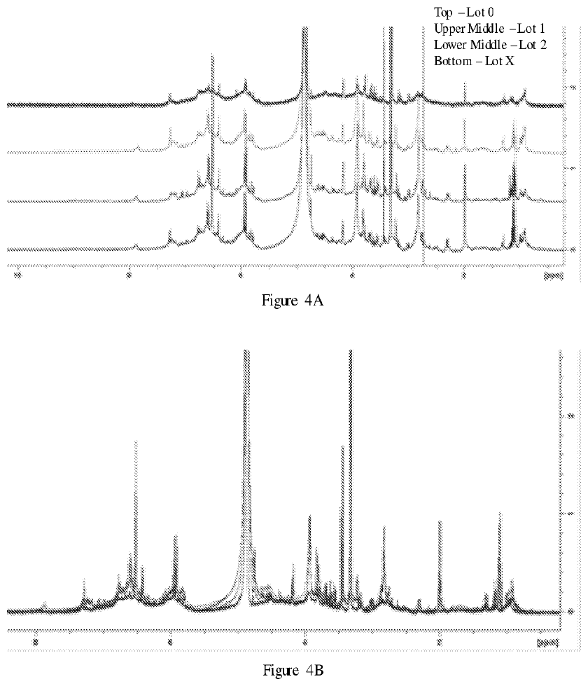

In another NMR analysis using the d₄-Methanol as the solvent, 4 distinct lots of AB-101 (Lots 00, 01, 02, and X respectively) are compared. NMR spectra of each lot are shown in FIG. 4A with overlays of each lots spectra being shown in FIG. 4B. While the fingerprint of the 4 lots looks similar, there are important differences. This is shown by comparing the concentration level in ppm based on LC-MS/MS Quantification and qualitative NMR “fingerprinting” on the marker compounds of catechin, epicatechin, gallocatechin, epigallocatechin, and taspine. The results are shown in Table 2 and indicate that lots 1 and 2 are more similar and lots X and 0 have the largest differences.

TABLE 2

| | AB-101 Lots Characterization | | | |
|---|---|---|---|---|
| | PPM (μg/g) | | | |
| Lot | X | 00 | 01 | 02 |
| Gallocatechin (GC) | 164.2 | 91.9 | 135.0 | 139.9 |
| Epigallocatechin (EGC) | 1357.6 | 380.7 | 1219.5 | 996.3 |
| Catechin (C) | 2.0 | 6.7 | 8.8 | 8.2 |
| Epicatechin (EC) | 2.6 | 5.2 | 8.3 | 6.1 |
| Taspine (T) | 50.4 | 43.4 | 50.1 | 51.1 |

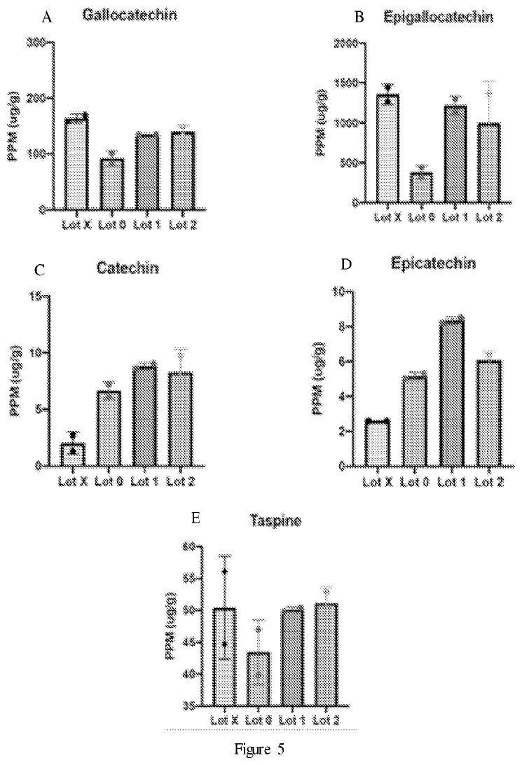

FIG. 5A-E depicts bar graphs comparing the AB-101 lot analysis results for each of the 5 marker compounds.

Lot X is an example of a lot that is not suitable for use in the pharmaceutical compositions and the methods of use described herein. Lots 00, 01 and 02 are examples of lots that are suitable for use in the pharmaceutical compositions and the methods of use described herein.

Some embodiments herein are directed to a pharmaceutical composition that further comprises one or more other therapeutic ingredients. In embodiments, the pharmaceutical composition comprises a therapeutically effective amount of the latex of Croton lechleri, preferably filtered latex of Croton lechleri, preferably filtered latex of Croton lechleri Müll.Arg. In embodiments, the pharmaceutical composition is suitable for topical administration or is a topical pharmaceutical composition.

The excipient(s) must be “acceptable” in the sense of being compatible with the other ingredients of the formulation and not deleterious to the recipient thereof. The excipient(s) will utilize a low number of known, well-characterized excipient ingredients that will not impart irritation or sensitization when used topically or in wounds or reduce the efficacy of AB-101. Proper formulation of the pharmaceutical composition is dependent upon the route of administration chosen. Any of the well-known techniques and excipients may be used as suitable and as understood in the art. The pharmaceutical compositions disclosed herein may be manufactured in any manner known in the art.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose, including eutectic solvents, eutectic-based ionic liquids, or ionic liquids. The pharmaceutical compositions can additionally include: lubricating agents such as talc, magnesium stearate, and mineral oil; wetting agents; emulsifying and suspending agents; preserving agents such as methyl- and propylhydroxy-benzoates.

The compositions include those suitable for topical (including, for example, dermal, nasal, oral mucosa, buccal, sublingual and intraocular) although the most suitable route may depend upon for example the condition and disorder of the recipient. The compositions may conveniently be presented in unit dosage form and may be prepared by any of the methods well known in the art of pharmacy. Typically, these methods include the step of bringing into association latex of Croton lechleri, preferably filtered latex of Croton lechleri, preferably filtered latex of Croton lechleri Müll.Arg. disclosed herein (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

The pharmaceutical compositions disclosed herein may be administered topically, that is by non-systemic administration. This includes the application of a composition disclosed herein externally to the surface of the skin and to achieve therapeutically effective amounts in the skin, such as the epidermis and/or dermis. In embodiments, topical administration or a topical pharmaceutical composition does not result in systemic administration or systemic exposure of the Croton lechleri to the patient.

In some embodiments, pharmaceutical compositions suitable for topical administration include liquid or semi-liquid preparations suitable for penetration through the skin to the site of inflammation such as a solution, powder, fluid emulsion, fluid suspension, semi-solid, ointment, paste, cream, gel, jelly, foam, liniment, lotion, and drops.

Lotions include those suitable for application to the skin. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid pharmaceutical compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base.

Preferred unit dosage pharmaceutical compositions are those containing an effective dose, as herein below recited, or an appropriate fraction thereof, of the active ingredient.

When employed as pharmaceuticals, the compositions can be administered in the form of pharmaceutical compositions. These compositions can be prepared in a manner well known in the pharmaceutical arts, and can be administered by a variety of routes, depending upon whether local or systemic treatment is desired and upon the area to be treated. Administration of the disclosed compositions may be topical (including dermal, nasal, oral mucosa, buccal, sublingual and intraocular). Pharmaceutical compositions for topical administration may include foams, transdermal patches, ointments, lotions, creams, gels, solutions, fluid emulsions, fluid suspensions, semi-solids, pastes, drops, suppositories, sprays, liquids, aerosolization, inhalers, and powders. Conventional pharmaceutical carriers, aqueous, powder or oily bases, thickeners and the like may be necessary or desirable. In some embodiments, the compositions can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance.

In certain embodiments, the pharmaceutical composition is not a soap.

In certain embodiments, the pharmaceutical composition is a liquid, ointment, lotion, or cream.

The pharmaceutical compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active pharmaceutical compositions comprising latex of Croton lechleri, preferably filtered latex of Croton lechleri, preferably filtered latex of Croton lechleri Müll.Arg. can be effective over a wide dosage range and contain therapeutically effective amount. It will be understood, however, that the amount of the pharmaceutical compositions comprising latex of Croton lechleri, preferably filtered latex of Croton lechleri, preferably filtered latex of Croton lechleri Müll.Arg. actually administered will usually be determined by a physician, according to the relevant circumstances, including the condition to be treated, the chosen route of administration, the actual composition administered, the age, weight, and response of the individual patient, the severity of the patient's symptoms, and the like.

The pharmaceutically acceptable excipient may be selected from one or more cream bases, one or more emulsifying agents, one or more preservatives, one or more humectants, one or more diluents, and latex of Croton lechleri, preferably filtered latex of Croton lechleri, preferably filtered latex of Croton lechleri Müll.Arg.

In some embodiments, the pharmaceutical composition may comprise about 0.01% to about 50% of latex of Croton lechleri, preferably filtered latex of Croton lechleri, preferably filtered latex of Croton lechleri Müll.Arg. disclosed herein. In some embodiments, the latex of Croton lechleri, preferably filtered latex of Croton lechleri, preferably filtered latex of Croton lechleri Müll.Arg. is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a composition made from AB-101 with exemplary amounts of the marker compounds present in the latex as disclosed in Table 1. To illustrate, a pharmaceutical composition comprising about 100% of AB-101 will contain at least about 85 PPM of gallocatechin, while a pharmaceutical composition comprising about 200% of AB-101 will contain at least about 170 PPM of gallocatechin. The foregoing all representing weight percentages of the pharmaceutical composition. In some embodiments, the pharmaceutical composition is suitable for topical administration (including, for example, dermal, nasal, oral mucosa, buccal, sublingual and intraocular).

In some embodiments, the latex of Croton lechleri, preferably filtered latex of Croton lechleri, preferably filtered latex of Croton lechleri Müll.Arg. is in a therapeutically effective amount. In some embodiments, the therapeutically effective amount may be in an amount of about 0.01% to about 100%, about 0.01% to about 95%, about 0.01% to about 90%, about 0.01% to about 85%, about 0.01% to about 80%, about 0.01% to about 75%, about 0.01% to about 70%, about 0.01% to about 65%, about 0.01% to about 60%, about 0.01% to about 55%, about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 100%, about 0.05% to about 95%, about 0.05% to about 90%, about 0.05% to about 85%, about 0.05% to about 80%, about 0.05% to about 75%, about 0.05% to about 70%, about 0.05% to about 65%, about 0.05% to about 60%, about 0.05% to about 55%, about 0.05% to about 50%, about 0.05% to about 45%, about 0.05% to about 40%, about 0.05% to about 30%, about 0.05% to about 20%, about 0.05% to about 10%, about 0.1% to about 100%, about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 300%, about 1% to about 295%, about 1% to about 290%, about 1% to about 285%, about 1% to about 280%, about 1% to about 275%, about 1% to about 270%, about 1% to about 265%, about 1% to about 260%, about 1% to about 255%, about 1% to about 250%, about 1% to about 245%, about 1% to about 240%, about 1% to about 235%, about 1% to about 230%, about 1% to about 225%, about 1% to about 220%, about 1% to about 215%, about 1% to about 210%, about 1% to about 205%, about 1% to about 200%, 195%, about 1% to about 190%, about 1% to about 185%, about 1% to about 180%, about 1% to about 175%, about 1% to about 170%, about 1% to about 165%, about 1% to about 160%, about 1% to about 155%, about 1% to about 150%, about 1% to about 145%, about 1% to about 140%, about 1% to about 135%, about 1% to about 130%, about 1% to about 125%, about 1% to about 120%, about 1% to about 115%, about 1% to about 110%, about 1% to about 105%, about 1% to about 100%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 2% to about 300%, about 2% to about 295%, about 2% to about 290%, about 2% to about 285%, about 2% to about 280%, about 2% to about 275%, about 2% to about 270%, about 2% to about 265%, about 2% to about 260%, about 2% to about 255%, about 2% to about 250%, about 2% to about 245%, about 2% to about 240%, about 2% to about 235%, about 2% to about 230%, about 2% to about 225%, about 2% to about 220%, about 2% to about 215%, about 2% to about 210%, about 2% to about 205%, about 2% to about 200%, 195%, about 2% to about 190%, about 2% to about 185%, about 2% to about 180%, about 2% to about 175%, about 2% to about 170%, about 2% to about 165%, about 2% to about 160%, about 2% to about 155%, about 2% to about 150%, about 2% to about 145%, about 2% to about 140%, about 2% to about 135%, about 2% to about 130%, about 2% to about 125%, about 2% to about 120%, about 2% to about 115%, about 2% to about 110%, about 2% to about 105%, about 2% to about 100%, about 2% to about 95%, about 2% to about 90%, about 2% to about 85%, about 2% to about 80%, about 2% to about 75%, about 2% to about 70%, about 2% to about 65%, about 2% to about 60%, about 2% to about 55%, about 2% to about 50%, about 2% to about 45%, about 2% to about 40%, about 2% to about 35%, about 2% to about 30%, about 2% to about 25%, about 2% to about 20%, about 2% to about 15%, about 3% to about 300%, about 3% to about 295%, about 3% to about 290%, about 3% to about 285%, about 3% to about 280%, about 3% to about 275%, about 3% to about 270%, about 3% to about 265%, about 3% to about 260%, about 3% to about 255%, about 3% to about 250%, about 3% to about 245%, about 3% to about 240%, about 3% to about 235%, about 3% to about 230%, about 3% to about 225%, about 3% to about 220%, about 3% to about 215%, about 3% to about 210%, about 3% to about 205%, about 3% to about 200%, 195%, about 3% to about 190%, about 3% to about 185%, about 3% to about 180%, about 3% to about 175%, about 3% to about 170%, about 3% to about 165%, about 3% to about 160%, about 3% to about 155%, about 3% to about 150%, about 3% to about 145%, about 3% to about 140%, about 3% to about 135%, about 3% to about 130%, about 3% to about 125%, about 3% to about 120%, about 3% to about 115%, about 3% to about 110%, about 3% to about 105%, about 3% to about 100%, about 3% to about 95%, about 3% to about 90%, about 3% to about 85%, about 3% to about 80%, about 3% to about 75%, about 3% to about 70%, about 3% to about 65%, about 3% to about 60%, about 3% to about 55%, about 3% to about 50%, about 3% to about 45%, about 3% to about 40%, about 3% to about 35%, about 3% to about 30%, about 3% to about 25%, about 3% to about 20%, about 3% to about 15%, about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 3%, about 5%, about 10%, about 15%, about 20%, about 25%, about 30%, about 35%, about 40%, about 45%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, or a range between any two of these values. The forgoing percentages are relative to a composition made from AB-101 with exemplary amounts of the marker compounds present in the latex as disclosed in Table 1. To illustrate, a therapeutically effective amount in the amount of about 100% of AB-101 will contain at least about 85 PPM of gallocatechin, while a therapeutically effective amount in the amount of about 200% of AB-101 will contain at least about 170 PPM of gallocatechin. The foregoing all representing weight percentages of the pharmaceutical composition.

In some embodiments, the therapeutically effective amount can vary according to, for example, the particular use for which the treatment is made, the manner of administration of the composition, the health and condition of the patient, and the judgment of the prescribing physician. The proportion or concentration of latex of Croton lechleri, preferably filtered latex of Croton lechleri, preferably filtered latex of Croton lechleri Müll.Arg. in a pharmaceutical composition can vary depending upon a number of factors including dosage, chemical characteristics (e.g., hydrophobicity), and the route of administration. For example, the compositions can be provided in an aqueous physiological buffer solution containing about 0.1 to about 10% w/v of the composition for parenteral administration. Some typical dose ranges for the compositions are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the composition selected, composition of the excipient, and its route of administration. Effective doses can be extrapolated from dose-response curves derived from in vitro or animal model test systems.

The amount of composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications.

In certain embodiments the one or more cream bases is selected from cetyl alcohol, isopropylmeristat, petroleum jelly, or any combination thereof. In some embodiments, the one or more cream bases is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more emulsifying agents is selected from span20, tween80, or any combination thereof. In some embodiments, the one or more emulsifying agents is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more preservatives is selected from propylparaben, methylparaben, or any combination thereof. In some embodiments, the one or more preservatives is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more humectants is propylene glycol. In some embodiments, the one or more humectants is in an amount of about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific examples may include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition. In some embodiments, the pharmaceutical composition is suitable for topical administration (including, for example, dermal, nasal, oral mucosa, buccal, sublingual and intraocular).

In certain embodiments the one or more diluents is water. Wherein the one or more diluents is in a quantity sufficient to bring the sum of the component weight percentages of the pharmaceutical composition to 100%.

The one or more ethanolic extracts of Croton lechleri is prepared by dissolving the latex of Croton lechleri, preferably filtered latex of Croton lechleri, preferably filtered latex of Croton lechleri Müll.Arg. tree in ethanol. The latex of Croton lechleri, preferably filtered latex of Croton lechleri, preferably filtered latex of Croton lechleri Müll.Arg. tree is not modified prior to dissolving in ethanol.

In some embodiments the pharmaceutical composition comprises about 10.0% cetyl alcohol, about 7.0% isopropylmeristat, about 21.0% petroleum jelly, about 1.5% span20, about 1.5% tween 80, about 0.02% propylparaben, about 0.18% methylparaben, about 5% propylene glycol, about 15% one or more ethanolic extracts of Croton lechleri, and water in a quantity sufficient to bring the sum of the component weight percentages of the pharmaceutical composition to 100%.

### Methods of Use

The present invention relate to methods of treatment of precancerous or cancerous skin lesions in a subject comprising the administration of a therapeutically effective amount of latex of Croton lechleri, preferably filtered latex of Croton lechleri, preferably filtered latex of Croton lechleri Müll.Arg. or a pharmaceutical composition containing latex of Croton lechleri, preferably filtered latex of Croton lechleri, preferably filtered latex of Croton lechleri Müll.Arg. as disclosed herein. In embodiments, the pharmaceutical composition may include a pharmaceutically acceptable excipient. As disclosed herein the latex of Croton lechleri, preferably filtered latex of Croton lechleri, preferably filtered latex of Croton lechleri Müll.Arg. shall comprise one or more compounds selected from: gallocatechin, epigallocatechin, catechin, epicatechin, and taspine, and combinations thereof. Each of gallocatechin, epigallocatechin, catechin, epicatechin, and taspine may be present in the latex of Croton lechleri, preferably filtered latex of Croton lechleri, preferably filtered latex of Croton lechleri Müll.Arg. in the amounts found in Table 1 or any combination of such amounts.

Also provided herein is latex of Croton lechleri, preferably filtered latex of Croton lechleri, preferably filtered latex of Croton lechleri Müll.Arg. as disclosed herein for use as a medicament.

Also provided herein is latex of Croton lechleri, preferably filtered latex of Croton lechleri, preferably filtered latex of Croton lechleri Müll.Arg. as disclosed herein for use as a medicament for the treatment of precancerous or cancerous skin lesions.

Also provided is the use of latex of Croton lechleri, preferably filtered latex of Croton lechleri, preferably filtered latex of Croton lechleri Müll.Arg. as disclosed herein as a medicament for the treatment of precancerous or cancerous skin lesions.

Also provided is latex of Croton lechleri, preferably filtered latex of Croton lechleri, preferably filtered latex of Croton lechleri Müll.Arg. latex of Croton lechleri Müll.Arg. as disclosed herein for use in the manufacture of a medicament for the treatment of precancerous or cancerous skin lesions.

Also provided is the use of latex of Croton lechleri, preferably filtered latex of Croton lechleri, preferably filtered latex of Croton lechleri Müll.Arg. as disclosed herein for the treatment of precancerous or cancerous skin lesions.

Also provided is the use of latex of Croton lechleri, preferably filtered latex of Croton lechleri, preferably filtered latex of Croton lechleri Müll.Arg. as disclosed herein

Also provided herein is a method of treating precancerous or cancerous skin lesions comprising contacting precancerous or cancerous skin lesions with latex of Croton lechleri, preferably filtered latex of Croton lechleri, preferably filtered latex of Croton lechleri Müll.Arg. as disclosed herein.

Also provided herein is a method for achieving a therapeutic effect in a patient comprising the administration of a therapeutically effective amount of latex of Croton lechleri, preferably filtered latex of Croton lechleri, preferably filtered latex of Croton lechleri Müll.Arg.

In some embodiments, the precancerous or cancerous skin lesion is a stage 0 cancer, In some embodiments, the precancerous or cancerous skin lesion is a stage I cancer, In some embodiments, the precancerous or cancerous skin lesion is a stage II cancer, In some embodiments, the precancerous or cancerous skin lesion is a stage III cancer, In some embodiments, the precancerous or cancerous skin lesion is a stage IV cancer.

In certain embodiments, the precancerous or cancerous skin lesion is selected from the group consisting of Non-hypertrophic Actinic Keratosis (AK), Superficial Basal Cell Carcinoma, Nodular Basal Cell Carcinoma, Superficial Squamous Cell Carcinoma, Squamous Cell Carcinoma, Squamous Cell Carcinoma in situ, Mycosis Fungoides and combinations thereof.

In certain embodiments, the precancerous or cancerous skin lesion is selected from the group consisting of Non-hypertrophic Actinic Keratosis, Superficial Basal Cell Carcinoma, Nodular Basal Cell Carcinoma, and Squamous Cell Carcinoma.

In certain embodiments, the precancerous skin lesion is Non-hypertrophic Actinic Keratosis (AK).

In certain embodiments, the cancerous skin lesion is selected from the group consisting of Superficial Basal Cell Carcinoma, Nodular Basal Cell Carcinoma, Superficial Squamous Cell Carcinoma, Squamous Cell Carcinoma, Squamous Cell Carcinoma in situ, and combinations thereof.

In certain embodiments the precancerous or cancerous skin lesion is not melanoma.

In certain embodiments the precancerous or cancerous skin lesion is not Mycosis Fungoides.

The pharmaceutical compositions may be administered in various modes, e.g. topical (including, for example, dermal, nasal, oral mucosa, buccal, sublingual and intraocular). Also, the route of administration may vary depending on the condition and its severity.

Pharmaceutical compositions of the present invention may be administered once per day, twice per day, thrice per day, 4 times per day, 5 times per day, 6 times per day, 7 times per day, 8 times per day, 9 times per day, 10 times per day, or a range between of these values. In some embodiments, the pharmaceutical compositions is administered twice per day. In some embodiments, the pharmaceutical composition is administered thrice per day. In some embodiments, the pharmaceutical composition is administered until the precancerous or cancerous skin lesion is resolved, gone, or treated.

Pharmaceutical compositions of the present invention may be administered continuously, every 15 minutes, 30 min, 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the administration lasts 12 weeks. In some embodiments the administration lasts until the precancerous or cancerous skin lesion is resolved, gone, or treated.

Treatment of the precancerous or cancerous skin lesions will last 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, or a range between of these values. In some embodiments, the treatment lasts 12 weeks.

Treatment of the precancerous or cancerous skin lesions may continue until complete resolution of the target lesion. In some embodiments the administration lasts until the precancerous or cancerous skin lesion is resolved, gone, or treated.

Treatment of the precancerous or cancerous skin lesions may continue at the discretion of the prescribing physician.

In certain embodiments, the pharmaceutical compositions of the present invention may be topically applied directly to the precancerous or cancerous skin lesions.

In certain embodiments, dosage is 1-2 drops of a pharmaceutical compositions of the present invention per precancerous or cancerous skin lesion, once, twice or more daily with the composition applied to each precancerous or cancerous skin lesion. Multiple drops are applied to a crop of lesions. The drops are allowed to dry (several minutes) or they are gently rubbed (about 15 seconds) over the precancerous or cancerous skin lesion until the composition changes to a “creamier” state. It then dries very quickly (several seconds).

In certain embodiments, the pharmaceutical compositions of the present invention is first applied to a bandage (e.g., gauze), which is then applied to the precancerous or cancerous skin lesion. The treated bandage is applied to each lesion. If the bandage is separated from the lesion or if the dressing has been worn for 24 hours, a new, treated bandage may be applied. A new dressing is generally, but not always, applied every day and may be applied up to once per week or longer period of time. In one embodiment, the composition is administered until the symptoms (e.g., skin lesions) disappear, become less pronounced, or problematic side effects occur.

In another embodiment, the pharmaceutical compositions can further include one or more additional pharmaceutical agents, such as a chemotherapeutic agent.

The pharmaceutical compositions of the present disclosure may be used to prevent or treat a precancerous or cancerous skin lesion by the sequential or co-administration of another pharmaceutical agent.

The compositions of the present invention can be used, alone or in combination with other pharmaceutically active agents, to treat conditions such as those previously described above. The compositions of the present invention and other pharmaceutically active agent(s) can be administered simultaneously (either in the same dosage form or in separate dosage forms) or sequentially. Accordingly, in one embodiment, the present invention comprises methods for treating a condition by administering to the subject a therapeutically-effective amount of one or more compositions of the present invention and one or more additional pharmaceutically active agents.

In certain instances, it may be appropriate to administer at least one of the compositions described herein, in combination with another pharmaceutical agent. Or, by way of example only, the therapeutic effectiveness of one of the compositions described herein may be enhanced by administration of an adjuvant (i.e., by itself the adjuvant may only have minimal therapeutic benefit, but in combination with another pharmaceutical agent, the overall therapeutic benefit to the patient is enhanced). Or, by way of example only, the benefit of experienced by a patient may be increased by administering one of the compositions described herein with another pharmaceutical agent (which also includes a therapeutic regimen) that also has therapeutic benefit. By way of example only, in a treatment for diabetes involving administration of one of the compositions described herein, increased therapeutic benefit may result by also providing the patient with another pharmaceutical agent for diabetes. In any case, regardless of the disease, disorder or condition being treated, the overall benefit experienced by the patient may simply be additive of the two pharmaceutical agents or the patient may experience a synergistic benefit.

Specific, non-limiting examples of additional pharmaceutical agents include use of compositions of embodiments herein with:

### Example 1

Objective: The objective of this clinical study is to determine whether topical application of AB-101 (a pharmaceutical composition of the present application) is safe and effective in the treatment of benign, precancerous and cancerous skin lesions.

Study Design: Up to 35 eligible participants age 18 or older will be enrolled and directed to self-administer AB-101 to each qualifying lesion twice per day (in the morning and at bedtime), after washing each affected area. For each lesion, 1 small drop of AB-101 was placed on each lesion, and rubbed gently in a circular motion until the material begins to lighten in color and becomes sticky/tacky. Once the material has turned to the lighter/stickier “foam”, it will dry within 10-20 seconds. If a patient washed the affected area within 2 hours of application, they were instructed to reapply AB-101. The application course will be for a period of up to 12 weeks. Participants will be evaluated weekly for the first 2 weeks of application, then at Weeks 4, 6, 8 and 12 for the duration of the application period.

Study Population: Male and female participants aged 18 and older with a clinical diagnosis of one of the following with no other meaningful uncontrolled systemic diseases can participate in this study.
Lentigines
Non-hypertrophic Actinic Keratosis (AK)
Superficial Basal Cell Carcinoma
Nodular Basal Cell Carcinoma
Superficial Squamous Cell Carcinoma
Squamous Cell Carcinoma in situ
Melanoma in situ (non-invasive)

Women must be either post-menopausal, non-pregnant, non-lactating, or practicing acceptable contraception. Women of childbearing potential, even if practicing a medically acceptable form of birth control, must have a negative urine pregnancy test prior to receiving study intervention, and agree to use a medically acceptable form of birth control, defined as hormonal (oral, implantable, or injectable) or mechanical (spermicide in conjunction with a barrier such as condom or diaphragm, IUD) contraceptives, during the study.

Hypothesis, Objective and Endpoints. The primary objective of this study is to determine whether topical application of AB-101 is effective in the treatment of benign, precancerous or cancerous skin lesions. The secondary objective of this study is to determine whether topical application of AB-101 is safe in the treatment of benign, precancerous or cancerous skin lesions.

Study Design. This is an open label study to evaluate the safety and effectiveness of AB-101 in participants with benign, precancerous or cancerous skin lesions. Up to 35 participants with a qualifying clinical diagnosis will be enrolled and directed to self-administer AB-101 to each qualifying lesion 2 times per day. The application course will be for a period of up to 12 weeks. Participants will be evaluated weekly for the first 2 weeks of application, then Week 4, 6, 8 and 12 for the duration of the application period.

Efficacy Evaluation.

Pre-treatment and post-treatment partial Biopsy in basal cell carcinoma, squamous cell carcinoma and Melanoma participants; Post-treatment Biopsy

Photography on iPad

Skin cancer history, concomitant medications, previous medications

Participant diary to track application, other medications

Study outcomes:
Subjective physician global assessment: complete, partial, or no resolution of lesions present at baseline
Objective measurement of lesion site
Lesion response at Weeks 1, 2, 4, 6, 8 and 12

Safety Evaluation. Safety will be evaluated by an analysis of adverse events.

Scientific Rationale for Study Design. This is an open-label study with no control arm.

Justification for Dose/Study Intervention Administered. In the current study AB-101 is used at 100% concentration as obtained in nature from the source. 100% AB-101 liquid is the study product. AB-101 is isolated from the resin and bark of several fast growing, medium to large trees found in the upper Amazon region.

Treatment Administration. The study treatment should be applied to the designated lesion twice daily. The participant will receive a Participant Diary at the Day 1 visit, which they will use to record their daily applications of the study treatment.

Efficacy Assessment—Lesion Response. Tumor Biopsy—from the following lesion types only:

Nodular Basal Cell Carcinoma

Superficial Basal Cell Carcinoma

Superficial Squamous Cell Carcinoma

Squamous Cell Carcinoma in situ

Melanoma in situ

A tissue biopsy will be obtained from the target lesion in order to confirm the clinical diagnosis at the Screening or Day 1 visit.

The effectiveness of AB-101 is confirmed by demonstrating a treatment response which would be either complete clearing of the cancer or a partial response. Even a stable response, where the cancer did not increase in size, can be viewed as a positive outcome, even though this is of a lesser degree.

Initial Lesion Measurement. The lesion area (length×width) will be measured and recorded. If a biopsy is obtained, lesion area will be measured after the biopsy is obtained.

Lesion Assessment During the Study. There were two measurements used to determine the response. For the first measurement, the investigator will record their assessment of the lesion response of each lesion at each study visit. The scale used for the Objective Response (OR) is shown below. The Objective Response Rate (ORR) is based on the observed changes between each visit. Lesion response is defined as:

Complete Response—Complete resolution of target lesion

Partial Response—Lesion reduced in size

Stable Disease—No change in lesion size from baseline

Progressive Disease—Lesion increased in size from baseline

Unevaluable (Participant Lost to Follow-up)

Any new lesions should be noted.

Lesion Measurement. The second measurement is lesion area. The lesion area (length×width) will be measured and recorded at each visit. Percentage change in area was calculated based on the change in area between visits. Area measurement for AK and Lentigines are not relevant measures so no measurements on these conditions were taken. Historically, the multiplicative product of the 2 longest diameters has also been used, with a ≥50% reduction in the product as evidence of a PR. Comparisons were made between the OR, ORR and % change in area were compared to confirm consistency.

Photography. A photograph of the target lesion will be obtained at each visit.

Safety Assessments (Adverse Events).

Adverse Events, Serious Adverse Events and Other Reportable Safety Events.

All adverse events occurring while the participant is receiving protocol therapy and during the follow-up period will be recorded. During the follow-up period, resolution of any adverse events present at the last application visit as well as any new adverse events will be recorded. Adverse events recorded at the last visit will require a phone call four weeks after the visit to determine resolution. All medications taken while the participant is receiving protocol therapy will be recorded. Completion of any medication regimen ongoing during the application period will also be recorded.

Fourteen subjects with 18 actinic keratosis (AK) clusters, 2 lentigines (LEN), and 19 skin carcinomas were enrolled and treated in protocol AB-101 at a single site. AB-101 usage across all patients included products from both lot 0 and lot X. The combined results across both lots show for the first time that AB-101 is an effective treatment for AKs, BCC and SCC. Since melanoma (MEL) is a serious cancer, and there was no response, the melanoma arm was discontinued because of the health risk. Also, the lentigines arm was suspended because after complete treatments, no changes were seen. While lentigines are not a serious skin condition, there was no reason to continue this arm if no changes were seen even in a couple of patients. Results are shown in Table 3.

TABLE 3

| | | Reduction | ORR |
|---|---|---|---|
| Lesion Type | # Lesions | (%) L × W | (%) |
| AK | 18 | n/a | 50% |
| BCC | 14 | 58% | 50% |
| SCC | 4 | 56% | 75% |
| LEN | 2 | n/a | 0% |
| MEL | 1 | 0% | 0% |

Comparison between Lot X and Lot 0 was made due to the differences in the chemical composition. This difference showed for the first time that the compositional differences contribute to the performance efficacy of AB-101. Lot 0 showed increased performance over Lot X. This increase in performance can be attributed to the primary changes in gallocatechin, epigallocatechins, catechin, epicatechin and taspine. Results are shown in Table 4.

TABLE 4

| | Lesion | | |
|---|---|---|---|
| AB-101 Lot | Type | # Lesions | ORR |
| Lot X | AK | 14 | 45% |
| Lot 0 | AK | 4 | 100% |
| Lot X | BCC | 8 | 25% |
| Lot 0 | BCC | 6 | 83% |
| Lot X | SCC | 1 | 25% |
| Lot 0 | SCC | 3 | 75% |

A secondary outcome of safety was measured. In all cases no safety incidences were recorded for pain, inflammation, redness, scabbing, scarring, disfigurement, permanent change in skin color, nausea or hair loss. No observation or patient reporting irritation, hypersensitivity photosensitivity was obtained. This shows AB-101 has the properties of being both gentle and effective.

Benefits of AB-101 can be seen as develop a standalone treatment in any topical treatment form or as an adjunct therapy for use in combination with conventional treatment, whether topical creams or devices as a regime or added to conventional skin cancer treatment topical products. AB-101 can even be used as an early treatment prior to conventional treatment.

1. A method of treating a precancerous or cancerous skin lesion in a subject in need thereof comprising topically administering a therapeutically effective amount of a pharmaceutical composition containing filtered latex of Croton lechleri, wherein the Croton lechleri Müll.Arg contains at least about 85 PPM of Gallocatechin, at least about 300 PPM of Epigallocatechin, at least about 2.4 PPM of Catechin, at least about 3.1 PPM of Epicatechin, at least about 35 PPM Taspine.

2. The method of claim 1, wherein the Croton lechleri is Croton lechleri Müll.Arg.

3. The method of claim 1, wherein the precancerous or cancerous skin lesion is selected from Non-hypertrophic Actinic Keratosis, Superficial Basal Cell Carcinoma, Nodular Basal Cell Carcinoma, Superficial Squamous Cell Carcinoma, Squamous Cell Carcinoma, Squamous Cell Carcinoma in situ, and Mycosis Fungoides.

4. The method of claim 3, wherein the precancerous or cancerous skin lesion is selected from Non-hypertrophic Actinic Keratosis, Superficial Basal Cell Carcinoma, Nodular Basal Cell Carcinoma, and Squamous Cell Carcinoma.

5. The method of claim 1, wherein the pharmaceutical composition is a liquid, ointment, lotion or cream.

6. The method of claim 1 wherein the administration is until the precancerous or cancerous skin lesion is treated.

7. The method of claim 1, wherein the pharmaceutical composition is topically administered directly to the precancerous or cancerous skin lesion.

8. The method of claim 1, wherein the treatment lasts until the precancerous or cancerous skin lesion is treated.

9. The method of claim 1, wherein the pharmaceutical composition is a liquid, ointment, lotion, or cream.

10. The method of claim 1, wherein the pharmaceutical composition further comprises a pharmaceutically acceptable carrier.

11. A pharmaceutical composition comprising a therapeutically effective amount of filtered latex of Croton lechleri Müll.Arg, wherein the Croton lechleri Müll.Arg contains at least about 85 PPM of Gallocatechin, at least about 300 PPM of Epigallocatechin, at least about 2.4 PPM of Catechin, at least about 3.1 PPM of Epicatechin, and at least about 35 PPM Taspine and wherein the pharmaceutical composition is suitable for topical administration.

12. The pharmaceutical composition of claim 11, wherein the pharmaceutical composition in a is a liquid, ointment, lotion, or cream.

13. The pharmaceutical composition of claim 12, wherein the therapeutically effective amount 3 to 100 wt %.

14. The pharmaceutical composition of claim 13, wherein the pharmaceutical composition further comprises a pharmaceutically acceptable carrier.
